(19) United States
(12) Patent Application Publication
(10) Pub. No.: US 2025/0243438 A1
(43) Pub. Date: Jul. 31, 2025

(54) PHOTOBIOREACTOR FOR THE CULTURE OF MACRO OR MICROORGANISMS, LIQUID EVAPORATION OR LIQUID FERMENTATION

(71) Applicant: BLUEMATER, S.A., PORTO (PT)

(72) Inventor: Nuno Horácio FERREIRA GOMES, SENHORA DA HORA (PT)

(21) Appl. No.: 18/705,086

(22) PCT Filed: Sep. 27, 2022
(86) PCT No.: PCT/IB2022/059191

§ 371 Date: Apr. 26, 2024

(30) Foreign Application Priority Data
Oct. 29, 2021 (PT) 117536

(51) Int. Cl. C12M 1/00

(52) U.S. Cl. CPC C12M 21/02; C12M 23/50; C12M 23/58; C12M 29/06; C12M 31/02

(57) ABSTRACT

A photobioreactor (1) for the culture of macro or microorganisms such as algae, fungi or bacteria, with further applications in the treatment of liquids, capture and mitigation of CO2 and greenhouse gases, processes of liquid evaporation or liquid fermentation. The photobioreactor (1) comprises two sections, an upper section (2) and a lower section (3), in which the upper section (2) comprises a front face (4) with a slope angle that varies between 0° and 90°, to arrange the front face (4) perpendicularly to the incident sunlight when the sun is in its zenith.

### TECHNICAL FIELD

This application relates to a photobioreactor for the culture of macro or microorganisms, liquid evaporation, or liquid fermentation.

### BACKGROUND ART

Currently, there are two types of photobioreactors: closed bioreactors and open raceway tanks.

Closed reactors are usually tubular, presenting horizontal or vertical orientation and having application problems such as low productivity, expensiveness, or construction difficulty and the impossibility to make a suitable industrial scale-up for large dimension units. Another variant of these reactors consists of plastic bags which, although present a high productivity per volume unit, have low productivity per unit of occupied area because of its low volume capacity, increasing the costs of installation and operation, and making it also uninteresting for industrial large-scale. This type of reactor is already used industrially in small units of a few hectares to produce high-value substances such as proteins, β-carotenes, astaxanthin, among others, which have high market value. However, the most promising markets for this technology are biofuels, large-scale biomass production, industrial CO₂ fixation, bioremediation, water treatment, and water distillation. To attend these applications a large-scale production is required, which cannot be achieved by the current technology in the state of the art.

As for the raceway tanks, they are open reactors that have low installation costs, but at the same time also have low productivity due to problems of poor insolation of the water column or contamination of the culture by bacteria, protozoa, and fungi. These problems make it uninteresting for intensive algae cultivation and for situations in which the product to be obtained presents low economic value, as, for example, the case of oils for biodiesel. However, given the lack of alternatives they are the most used systems nowadays.

The algae culture is at a point where there is an emerging market, but the existing cultivation solutions do not satisfy it from a productive and commercial point of view.

### SUMMARY

The present invention relates to a photobioreactor (1) for the culture of macro or microorganisms, liquid evaporation or liquid fermentation comprising:
an upper section (2) and a lower section (3) joined to each other by fixing means (12) and further comprising an o-ring (13) inserted on the periphery of the lower section (3); and
the upper section (2) further comprises a front face (4);
at least one lower inlet (5.1) and at least one lower outlet (5.2) are arranged in the lower section (3);
at least one upper inlet (8.1) and at least one upper outlet (8.2) arranged on the upper section (2);
a removable supply tube (6) arranged on the periphery of the lower section (3) with gas outlet holes (7) arranged on the length of the supply tube (6);
an upper face (2.1) adjacent to the front face (4), and at least one opening (10) and at least one intermediate hole (11);
   and the front face (14) is arranged in an angle (15) that varies between 0° and 90°, which is defined by the inclination at the point of intersection of the front face (4) with a horizontal axis (H) that is parallel to the lower section (3).

In one embodiment the fixing means (12) is selected from springs, staples, screws, glue, thermo-gluing.

In one embodiment the photobioreactor (1) is made from a transparent synthetic material.

In one embodiment the lower section (3) is made of a metal.

In one embodiment the upper section (2) is made of a transparent material selected from polyacrylate, polycarbonate or polyethylene.

In one embodiment the upper inlet (8.1) and upper outlet (8.2) are of the overflow type.

In one embodiment the openings (10) comprise covers (23).

In one embodiment the front face (4) has a flat shape or has a curved shape.

In one embodiment the photobioreactor comprises a geometric shape such as cubic, parallelepiped, trapezoidal or prismatic shape.

In one embodiment the photobioreactor (1) further comprises at least one peripheral channel (21) and at least one lateral opening (22).

The invention further relates to a module of photobioreactors (24, 30) comprising a plurality of photobioreactors (1) and further comprising threaded parts and/or other fastening means (9).

The invention further relates to a sector of photobioreactors (25) comprising a plurality of modules (24) and further comprising inlet pipes (26) and outlet pipes (27).

The invention further relates to a field of photobioreactors (28) comprising a plurality of sectors (25) and further comprising inlet pipes (26) and outlet pipes (27).

The invention further relates to a field of vertical photoreactors (29) comprising a plurality of modules (24, 30) supported on structures (31) and further comprising inlet pipes (26) and outlet pipes (27).

### GENERAL DESCRIPTION

The proposed biological photobioreactor of the present application is a new type of bioreactor that aims to improve the cultivation conditions to increase the productivity of microalgae, macroalgae, and bacteria to obtain biomass that can be transformed into value added products. The photobioreactor can also be used for processes that involve the evaporation of liquids or the fermentation of liquids.

This photobioreactor can be used for several purposes, the most important being:
Cultivating microalgae to obtain oils that can be used to produce biodiesel, proteins, β-carotenes, sugars, and other substances of interest for human consumption, cosmetics, and the agri-food industry;
Cultivating microalgae for the treatment of wastewater and other concentrated effluents, such as leachate from landfills or industrial processes;
Cultivating macroalgae to produce agarose, carrageenan and other substances of interest for the culinary, and pharmaceutical industry;
Cultivating cyanobacteria and other microorganisms for the production of hydrogen;
Cultivating microalgae, macroalgae, and cyanobacteria for the fixation of industrial carbon dioxide, and environmental bioremediation;
Cultivating bacterial culture by aerobic, and fermentative processes;
Other fermentative processes with bacteria and fungi;
Desalting, purification or treatment of liquids, such as water;
Fermentation processes.

The design of the photobioreactor considers seven key aspects:
a) Format—the shape of the photobioreactor is designed to optimize volume and sun exposure, both key aspects for a good microalgae reactor;
b) Dimension—to reduce manufacturing, transport, and installation costs, the photobioreactor should be as large as possible, limited only by the materials and moulds available for its manufacture and made in two parts for stacked transport;
c) Upper face slope—needs to be adequate to the latitude where the photobioreactor will be implemented and shall be perpendicular to the incident sunlight when the sun is at its zenith during the equinoxes;
d) Materials—robust and reliable materials capable of maintaining a longevity of not less than 10 years, to minimize production costs and maintain transparency throughout life;
e) Modularity—it should allow the construction of modules with several photobioreactors in an easy and modular way, to obtain modules, sectors and fields of photobioreactors necessary for an adequate water treatment system or culture of microalgae and other microorganisms;
f) Automation and control—individual photobioreactors, modules, sectors and fields of photobioreactors can be automated through an automation and control system.
g) Manufacturing and logistics—optimized format for transport in overlapping modules, saving space and costs of logistics.

The photobioreactor of the present application can be applied in the following scenarios:
a) Cultivation of microalgae, macroalgae, bacteria, cyanobacteria, and fungi to produce added value substances in their autotrophic, heterotrophic or mixotrophic phase;
b) Treatment of urban and industrial wastewaters, landfill and other leachates, aquaculture and public aquariums recirculating waters, and eutrophic waters of ponds, lakes and other surface waters;
c) Capture and mitigation of CO₂ and other Greenhouse gases (GHG) emitted by industrial units, power plants, refineries and other GHG emission sources;
d) Fermentative processes to obtain alcohols, methane, hydrogen and other valuable substances;
e) Evaporation processes to obtain distilled water from saltwater or brackish water, leachates, wastewaters, and other liquids that aims to separate water from the suspended and dissolved components, to evaporate and collect alcohol, methane, hydrogen and other valuable substances from the liquid phase source, and in cooling or eating processes of water and other liquids trough the solar energy.

### BRIEF DESCRIPTION OF DRAWINGS

For easier understanding of this application, figures are attached in the annex that represent the preferred forms of implementation which nevertheless are not intended to limit the technique disclosed herein.

FIG. 1 shows a perspective view of the photobioreactor (1).

FIG. 2 shows a perspective view of the photobioreactor (1) with all technical features.

FIG. 3 shows an example of different solar incidences (14) on the front face (4) of the photobioreactor (1), depending on the latitude in which it is installed, the period of the year, time of day and orientation of the photobioreactor in relation to the sun.

FIG. 4 shows a side view of the photobioreactor (1) showing the upward flow movement of gases (16) from the supply tube (6).

FIG. 5 shows a side view of the photobioreactor (1) showing the medium liquid (18) upward flow movement of evaporated liquid (19), condensation (20) of the evaporated liquid at the inner walls of the upper section (2).

FIG. 6 shows a perspective view of several photobioreactors (1) interconnected by fastening means (9), forming a module of photobioreactors (24).

FIG. 7 shows a perspective view of a sector of photobioreactors (25), which are connected to each other in series and/or parallel by inlet pipes (26) and outlet pipes (27).

FIG. 8 shows a field of photobioreactors (28) constituted by several sectors of photobioreactors (25) constituted by several modules of photobioreactors (24).

FIG. 9 shows a perspective view of a field of vertical photobioreactors (29).

FIG. 10 shows a side view of the two main sections of the photobioreactor (2, 3), stacked for transport.

### DESCRIPTION OF EMBODIMENTS

Now, preferred embodiments of the present application will be described in detail with reference to the annexed drawings. However, they are not intended to limit the scope of this application.

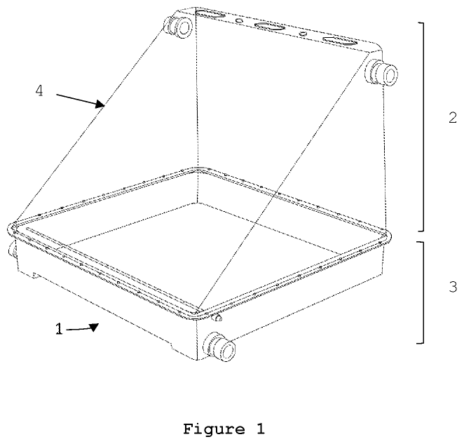

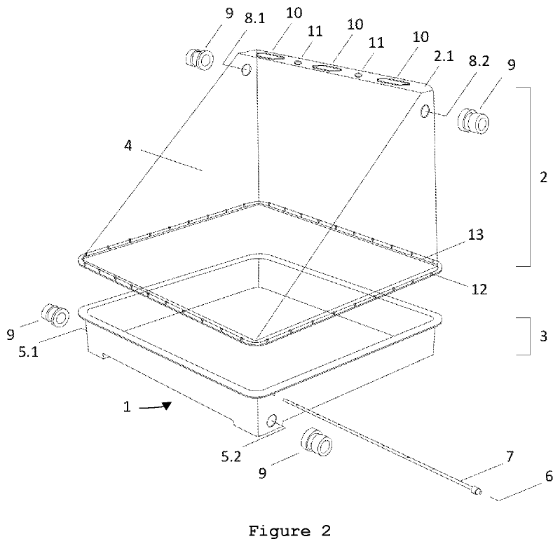

This photobioreactor (1) comprises two sections: an upper section (2) and a lower section (3) as shown in FIGS. 1 and 2. These sections are joined to each other by fixing means (12) that can be selected from springs, staples, screws, glue, thermo-gluing, or any other fixing means suitable to connect the upper section (2) to the lower section (3). The upper section (2) further comprises a front face (4).

The photobioreactor (1) of the present invention as shown in FIGS. 1 and 2, can be made from a transparent synthetic material. The selected material is preferably one that is resistant to environmental temperature variations.

The upper section (2) can be made in a transparent material selected from, but not limited to, polyacrylate, polycarbonate or polyethylene terephthalate plastic.

The lower section (3) can be made in the same material of the upper section (2), but transparency is not the most important property due to the relative position to the sun. Rather, the most important feature is to be resistant to the traction and weight of liquids. Optionally, the materials used for this lower section (3) can be reinforced with fiberglass or other suitable fibers. Other materials to produce the lower section (3) include metals, for example cast aluminum.

The photobioreactor (1) further comprises an o-ring (13) inserted on the periphery of the lower section (3), as shown in FIG. 2, which is suitable to connect the upper section (2) and the lower section (3) and keep the photobioreactor (1) sealed.

The photobioreactor (1) further comprises at least one lower inlet (5.1) and at least one lower outlet (5.2) arranged in the lower section (3), which are suitable for inoculation and nutrient feeding and for harvesting or recirculation of liquid medium.

At least one upper inlet (8.1) and at least one upper outlet (8.2) are arranged on the upper section (2) of the photobioreactor (1), preferably on a lateral face of the upper section (2). The upper inlet and outlet (8.1, 8.2) are preferably of the overflow type. These upper inlet and outlet (8.1, 8.2) are suitable to avoid overfilling the photobioreactor (1) in cases of in-line operation or for top discharge when it is required to keep the photobioreactor (1) full and leveled by that way.

A supply tube (6) is arranged on the periphery of the lower section (3), i.e., the bottom edge of the front face (4), and is suitable to supply gas, such as air or CO₂. Gas outlet holes (7) are arranged on the length of the supply tube (6). The Gas outlet holes (7) are directed towards the internal side of the front face (4). The gas outlet holes (7) are suitable to direct gas towards the internal side of the front face (4) of the photobioreactor (1) and ensure the circulation of gas tangential to the front face (4) and the recirculation of liquid inside the photobioreactor (1). The gas outlet holes (7) are also suitable to perform the internal cleaning of internal side of the frontal face (4).

This supply tube (6) is removable for cleaning and any unclogging of the air and gas outlet holes (7) that may be necessary.

The photobioreactor (1) further comprises an upper face (2.1) adjacent to the front face (4). The upper face (2.1) comprises at least one opening (10) which is suitable to eliminate excess gases or guide water vapor to a condenser, or to ensure easy access to the interior of the photobioreactor (1). The upper face (2.1) further comprises at least one intermediate hole (11) suitable for the placement of probes to measure and control the photobioreactor (1).

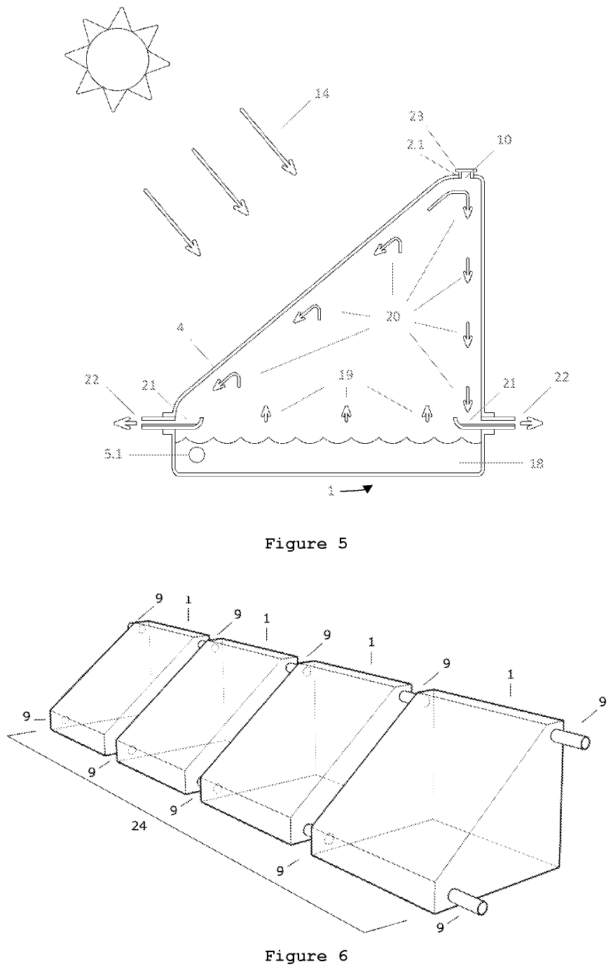

These openings (10) also provide an easy access to the interior of the photobioreactor (1) for cleaning, installation of aerators or connectors, or other required work. These openings (10) can comprise covers (23) suitable to prevent evaporation, as shown in FIG. 5. They can be watertight or not, made from the same material as the photobioreactor (1) or in rubber, cork, wood, or other suitable material.

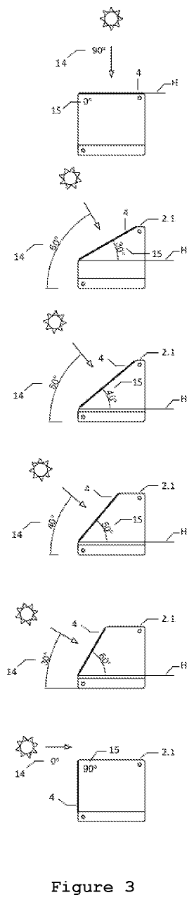

The front face (4) of the photobioreactor (1) is arranged towards the incident sunlight (14), as shown in FIGS. 3, 4 and 5.

The angle (15) of the front face (4) that is arranged towards the incident sunlight (14), as shown in FIG. 3, is variable and depends on the installation latitude of the photobioreactor (1), calculated to arrange the front face (4) perpendicularly to the incident sunlight (14) at the solar zenith. This front face's (4) angle (15) varies between 0° (equator) and 90° (the latitude of the polar poles) and is defined by the slope at the point of intersection of the front face (4) with a horizontal axis (H) that is parallel to the lower section (3) of the photobioreactor (1), as shown in FIG. 3. The purpose of this angle (15) variation is to obtain an optimum slope of the front face (4) arranged towards incident sunlight (14), which is perpendicular to the incident sunlight when the sun is at its zenith during the autumn and spring equinoxes (FIG. 3).

The angle (15) is calculated according to the latitude where the photobioreactor (1) is installed, to maximize the solar incidence that the front face (4) receives during the year. This same angle (15) can assume any value within the Cartesian quadrant facing the sun.

The front face (4) can have a flat shape or have a curved shape.

Thus, the solar exposure will be enhanced, minimizing the light losses by reflection and refraction (FIG. 3).

For example, in the latitude of Portugal this optimal slope angle would be approximately 40°, in the latitude of Stockholm it would be approximately 60° and in the latitude of Miami it should be approximately 26°.

This slope variation can be obtained by different inclination angles (15) of the front face (4) of the photobioreactor (1), allowing the photobioreactor (1) to assume geometric shapes such as cubic, parallelepiped, trapezoidal or prismatic shape (FIG. 3).

The shape of both sections was optimized for industrial production by injecting a material into a mould.

The upward circulation of gas bubbles (16) from the supply tube (6) assures the internal circulation (17) of the liquid medium (18), promoting the liquid rotating and the mixing of CO₂ and other gases injected into the liquid medium (18) and the dissipation of excess gases into the atmosphere, through the gas purge openings (10).

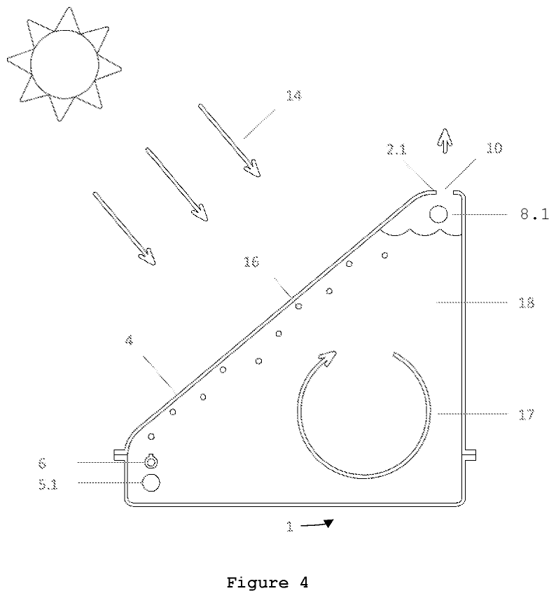

The formation of gas bubbles (16) also allows the regular contact of micro or macroorganisms that can be in the shadow zones of sunlight coming in through the front face (4), as shown in FIG. 4.

The air bubbles (16) also create points of concentration for the sunlight rays, since they act as lenses, causing the intermittence of the insolation inside the photobioreactor (1), which, in particular applications, stimulate the growth of photosynthetic organisms.

Optionally, the photobioreactor (1) can be equipped with a liquid recirculation pump and/or a heat exchanger or vapor refrigeration system.

FIG. 5 shows a side view of one embodiment of the photobioreactor (1) showing the liquid medium (18) with an upward flow movement of evaporated liquid (19), condensation (20) of the evaporated liquid (19) at the internal sides of the upper section (2), collecting said condensation (20) in at least one peripheral channel (21) and leading the condensed liquid through at least one lateral opening (22) suitable to collect the condensation (20).

Photobioreactor systems of different sizes can be created through a connection between a plurality of photobioreactors (1), through connection elements based on threaded parts and/or other fastening means (9), which will ensure the interconnection between them, thus constituting a module (24, 30), a sector (25) or field (28, 29) of photobioreactors (1) that can be interconnected by pipes (26, 27).

FIG. 6 shows a perspective view of several photobioreactors (1) interconnected by fastening means (9), such as pipes, or other fastening means through which several photobioreactors (1) can be connected in series, through which a cultured or evaporating liquid can circulate, forming a module of photobioreactors (24).

A module of photobioreactors (24) comprises a plurality of photobioreactors (1) and threaded parts and/or other fastening means (9).

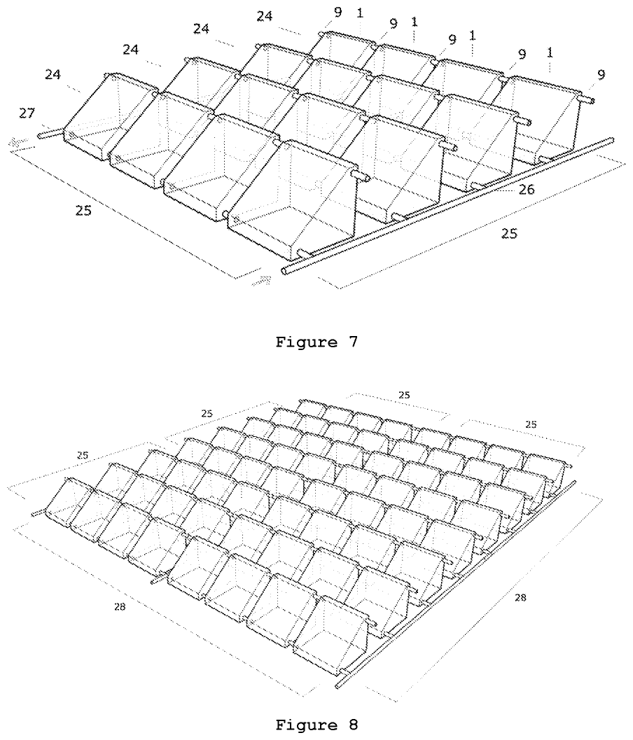

FIG. 7 shows a perspective view of a sector of photobioreactors (25) comprising a plurality of modules (24), in which the photobioreactors (1) are connected to each other in series and/or parallel by inlet pipes (26) and outlet pipes (27) to ensure lines of interconnected photobioreactors (i.e., modules (24)), which can be combined in sectors of photobioreactors (25). A plurality of sectors of photobioreactors (25) are able to form fields of photobioreactors (28). The sectors of photobioreactors (25) further comprise inlet pipes (26) and outlet pipes (27).

FIG. 8 shows a field of photobioreactors (28) comprising a plurality of sectors of photobioreactors (25) which in turn comprise several photobioreactor modules (24). This arrangement is suitable to use in different scales in water treatment, evaporation of liquids, culture of microalgae and other microorganisms, GHG capture, etc. The field of photobioreactors (28) further comprise inlet pipes (26) and outlet pipes (27).

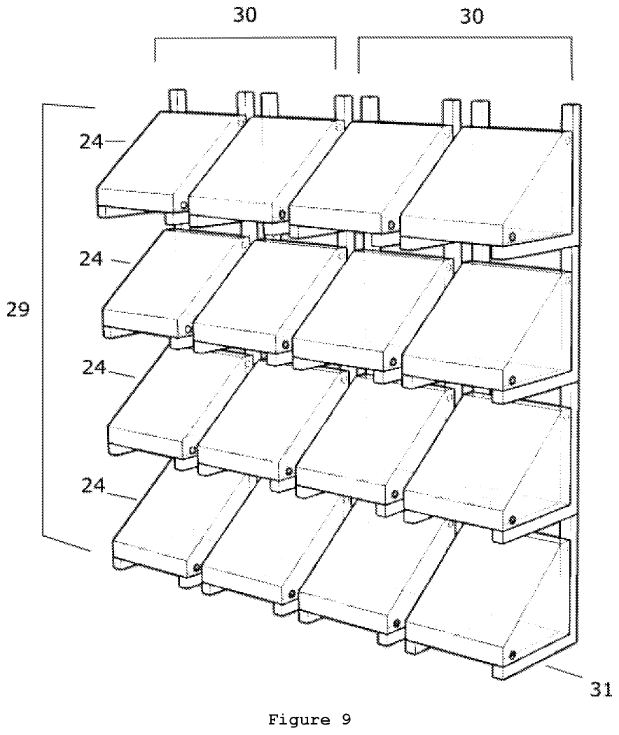

FIG. 9 shows a perspective view of a field of vertical photobioreactors (29) comprising a plurality of modules (24, 30), which are connected to each other in series and/or parallel to ensure lines of interconnected reactors, which can be combined in a plurality of sectors of a plurality of modules (30), which in turn can be combined in fields of vertical photobioreactors (29), supported on structures (31) and suitable for application in areas with lack of space which will be frequent for the use of this technology at different scales in water treatment, evaporation of liquids, culture of microalgae and other microorganisms, GHG capture, in industries, refineries and other polluting etc., structures or those intending to cultivate microorganisms in confined spaces. The field of vertical photobioreactors (29) further comprise inlet pipes (26) and outlet pipes (27).

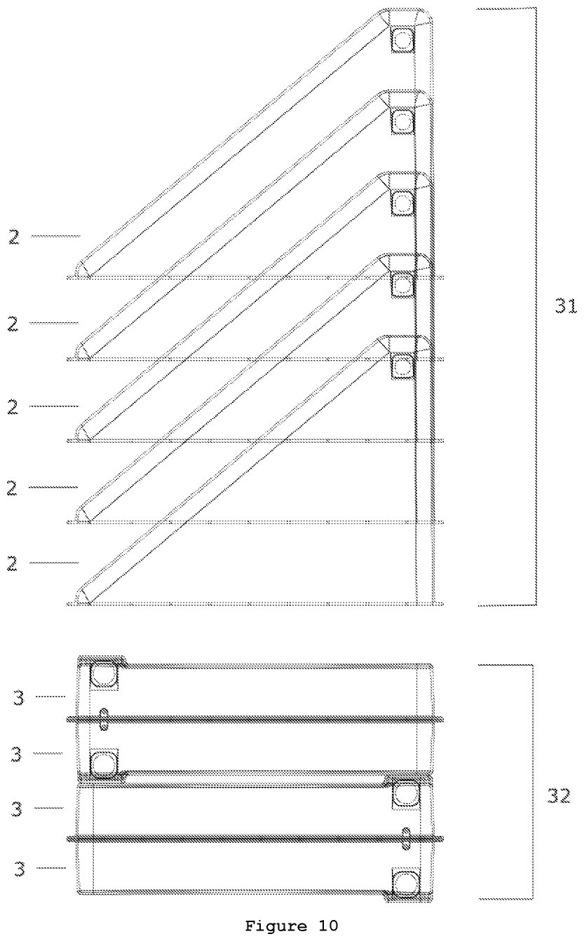

FIG. 10 shows a side view of the two main sections (2, 3) of the photobioreactor (1), stacked for transport. Stacked upper sections (31) are fitted together and stacked lower sections (32) are also fitted together, either arranged in opposite directions or fitted into each other to save space and thus allow to transport more photobioreactors (1) in containers or other means of transport in a manner that does not occupy much space.

These photobioreactors (1) can be applied in series or in parallel, to enhance the growth of micro or macro-organisms, fermentation or evaporation of liquids, depending on the purpose for which they are intended and the cultivation methods (FIGS. 6 to 9). If they are used to produce microalgae, they can work continuously, passing the algae culture from one to the other, with a removal rate by an automatic algae extraction system equal to the growth rate, or else work by the system of batch, which consists of filling and introducing the culture medium and the inoculum into the photobioreactor (1), with the microalgae being harvested at the end of the production cycle, when the photobioreactors are emptied. The modules (24) may, in turn, be organized into sectors (25, 30) each with the different culture inoculation time in the case of batch systems and interconnected in series or photobioreactor sectors in fields (28, 29). This allows even in batch the production of cultured microorganisms is continuous, by differentiated inoculation from homogeneous sectors of photobioreactors, depending on the time foreseen between inoculation and harvest. Both the individual photobioreactors (1), or the modules (24), sectors (25, 30) or fields (28, 29) of photobioreactors, may have automatic monitoring through parametric probes built into the photobioreactors, and automatically controlled, by the insertion of electric or pneumatic control valves or cut off the water flow and air or CO₂, commanded by parametric probes and connected to a supervision system.

### REFERENCE NUMBERS

1—Photobioreactor
2—upper section
2.1—upper face
3—lower section
4—front face
5.1—lower inlet
5.2—lower outlet
6—supply tube
7—gas outlet holes
8.1—upper inlet
8.2—upper outlet
9—fastening means
10—opening
11—intermediate holes
12—fixing means
13—o-ring
14—incident sunlight
15—angle
16—gas bubbles
17—circulation
18—liquid medium
19—evaporated liquid
20—condensation
21—peripheral channel
22—lateral opening
23—cover
24—module of photobioreactors
25—sector of photobioreactors
26—inlet pipes
27—outlet pipes
28—field of photobioreactors
29—field of vertical reactors
30—sectors of a plurality of modules
31—stacked upper sections
32—stacked lower sections
H—horizontal axis

This description is of course not in any way restricted to the forms of implementation presented herein and any person with an average knowledge of the area can provide many possibilities for modification m thereof without departing from the general idea as defined by the claims. The preferred forms of implementation described above can obviously be combined with each other. The following claims further define the preferred forms of implementation.

1. A photobioreactor (1) for the culture of macro or microorganisms, liquid evaporation or liquid fermentation, comprising:
an upper section (2) and a lower section (3) joined to each other by fixing means (12) and further comprising an o-ring (13) inserted on the periphery of the lower section (3); and
the upper section (2) further comprises a front face (4);
at least one lower inlet (5.1) and at least one lower outlet (5.2) are arranged in the lower section (3);
at least one upper inlet (8.1) and at least one upper outlet (8.2) arranged on the upper section (2);
a removable supply tube (6) arranged on the periphery of the lower section (3) with gas outlet holes (7) arranged on the length of the supply tube (6);
an upper face (2.1) adjacent to the front face (4), and at least one opening (10) and at least one intermediate hole (11);
and the front face (14) is arranged in an angle (15) that varies between 0° and 90°, which is defined by the inclination at the point of intersection of the front face (4) with a horizontal axis (H) that is parallel to the lower section (3).

2. The photobioreactor (1) according to claim 1, wherein the fixing means (12) are selected from springs, staples, screws, glue, or thermo-gluing.

3. The photobioreactor (1) according to claim 1, wherein the photobioreactor (1) is made from a transparent synthetic material.

4. The photobioreactor (1) according to claim 1, wherein the lower section (3) is made of a metal.

5. The photobioreactor (1) according to claim 1, wherein the upper section (2) is made of a transparent material selected from polyacrylate, polycarbonate or polyethylene.

6. The photobioreactor (1) according to claim 1, wherein the upper inlet (8.1) and the upper outlet (8.2) are of the overflow type.

7. The photobioreactor (1) according to claim 1, wherein the at least one opening (10) comprises a cover (23).

8. The photobioreactor (1) according to claim 1, wherein the front face (4) has a flat shape or a curved shape.

9. The photobioreactor (1) according to claim 1, wherein the photobioreactor (1) comprises a geometric shape.

10. The photobioreactor (1) according to claim 1, wherein it further comprises at least one peripheral channel (21) and at least one lateral opening (22).

11. A module of photobioreactors (24, 30) comprising a plurality of photobioreactors (1) as described in claim 1, and further comprising threaded parts and/or other fastening means (9).

12. A sector of photobioreactors (25) comprising a plurality of modules (24) as described in claim 11 and further comprising inlet pipes (26) and outlet pipes (27).

13. A field of photobioreactors (28) comprising a plurality of sectors (25) as described in claim 12 and further comprising inlet pipes (26) and outlet pipes (27).

14. A field of vertical photoreactors (29) comprising a plurality of modules (24, 30) as described in claim 11, supported on structures (31) and further comprising inlet pipes (26) and outlet pipes (27).

15. The photobioreactor according to claim 9, wherein the geometric shape is cubic, parallelepiped, trapezoidal or prismatic.
